(19) United States
(12) Patent Application Publication
(10) Pub. No.: US 2026/0358455 A1
(43) Pub. Date: Nov. 12, 2026

(54) MAGNETIC RESONANCE IMAGING APPARATUS, METHOD FOR OPERATING MAGNETIC RESONANCE IMAGING APPARATUS, AND PROGRAM

(71) Applicant: FUJIFILM Corporation, Tokyo (JP)

(72) Inventor: Shohei HATORI, Tokyo (JP)

(73) Assignee: FUJIFILM Corporation, Tokyo (JP)

(21) Appl. No.: 19/672,872

(22) Filed: May 11, 2026

(30) Foreign Application Priority Data
May 12, 2025 (JP) 2025-079647

(51) Int. Cl. G01R 33/563; A61B 5/055; G01R 33/56

(52) U.S. Cl. CPC G01R 33/56308; A61B 5/055; G01R 33/5608

(57) ABSTRACT

Provided is a magnetic resonance imaging apparatus that measure a blood velocity by applying a velocity encoding having an appropriate value according to a type of a blood vessel designated by a user. A magnetic resonance imaging apparatus according to the present disclosure causes a user to input a type of a target blood vessel before a measurement start operation by the user, measures a maximum blood velocity in a target blood vessel region, which is a region of the target blood vessel, from an image obtained in a pre-scan executed after the measurement start operation, sets a velocity encoding value for a main scan based on the maximum blood velocity, executes the main scan of a subject by applying a velocity encoding having the set value, and outputs an image showing the blood velocity in the target blood vessel region obtained in the main scan.

### CROSS-REFERENCE TO RELATED APPLICATION

The present application claims priority under 35 U.S.C § 119(a) to Japanese Patent Application No. 2025-079647 filed on May 12, 2025, which is hereby expressly incorporated by reference, in its entirety, into the present application.

### BACKGROUND OF THE INVENTION

### 1. Field of the Invention

The present disclosure relates to a magnetic resonance imaging apparatus, a method for operating a magnetic resonance imaging apparatus, and a program, and particularly to a vascular imaging technique based on a phase contrast method.

### 2. Description of the Related Art

In phase contrast (PC) magnetic resonance imaging (MRI) capable of imaging a velocity of blood flow (blood flow velocity), it is necessary to set a maximum value of the blood flow velocity in a blood vessel region to be imaged as a set value (VENC value) of velocity encoding (VENC) during main scan imaging.

As the VENC value, in a case in which a value extremely greater than the appropriate set value is set, a velocity noise ratio (VNR) is deteriorated. On the other hand, in a case in which a value smaller than the appropriate set value is set, velocity aliasing occurs, and an accurate blood flow velocity cannot be obtained.

Examples of a case in which an inappropriate VENC value is set include a case in which a blood vessel measured in a pre-scan executed to determine that the VENC value is incorrect, or a case in which a blood flow velocity is measured in a blood vessel deformed due to a disease or the like.

Multiple images with different VENC values may be performed on a plurality of blood flow velocities. In this case, the VENC values may be set individually for different blood vessels, or different values may be set for the same blood vessel.

### SUMMARY OF THE INVENTION

In a PC imaging method, a two-dimensional (2D) pre-scan is executed before a main scan, a blood flow velocity of a blood vessel to be imaged is measured from an image obtained in the pre-scan to set an appropriate VENC value, and then the main scan is executed. However, there is a problem in that an operation of a user is complicated and an incorrect parameter setting may also occur.

US9101283B discloses a method for automatically determining a VENC value in a pre-scan immediately before main imaging by simply selecting a target blood vessel on a blood flow image captured in advance, but there remains a complication that a user operation is required in the middle.

JP2000-175885A discloses a method for automatically determining a VENC value for imaging any one of an artery or a vein, but it is a technique of eliminating an artery or a vein by setting the VENC value to be larger than an appropriate value of a target blood vessel, and it is not possible to set an appropriate value for the target blood vessel.

The present disclosure has been made in view of such circumstances, and an object of the present disclosure is to provide a magnetic resonance imaging apparatus, a method for operating a magnetic resonance imaging apparatus, and a program that measure a blood velocity by applying a velocity encoding having an appropriate value according to a type of a blood vessel designated by a user.

In order to achieve the above object, a magnetic resonance imaging apparatus according to a first aspect of the present disclosure is a magnetic resonance imaging apparatus that measures a blood velocity of a blood vessel of a subject by applying a velocity encoding having a set value, the magnetic resonance imaging apparatus comprising: a processor, in which the processor is configured to: cause a user to input a type of a target blood vessel, which is the blood vessel to be measured, before a measurement start operation by the user; execute a pre-scan of the subject after the measurement start operation; detect a target blood vessel region, which is a region of the target blood vessel, from an image obtained in the pre-scan; measure a maximum blood velocity in the target blood vessel region from the image obtained in the pre-scan; set a velocity encoding value for a main scan based on the maximum blood velocity; execute the main scan of the subject by applying the velocity encoding having the set value; and output an image showing the blood velocity in the target blood vessel region obtained in the main scan.

According to a second aspect of the present disclosure, in the magnetic resonance imaging apparatus according to the first aspect, it is preferable that the processor is configured to: detect a blood vessel region, which is a region of a blood vessel, from the image obtained in the pre-scan; acquire a variation over time in blood velocity in the blood vessel region; and determine whether or not the blood vessel region is the target blood vessel region based on the variation in blood velocity.

According to a third aspect of the present disclosure, in the magnetic resonance imaging apparatus according to the first aspect or the second aspect, it is preferable that the processor is configured to: generate a three-dimensional image of a blood vessel from the image obtained in the pre-scan; and analyze the three-dimensional image to extract the target blood vessel region.

According to a fourth aspect of the present disclosure, in the magnetic resonance imaging apparatus according to any one of the first aspect to the third aspect, it is preferable that the processor is configured to set the velocity encoding value for the main scan for each of a plurality of the target blood vessels.

According to a fifth aspect of the present disclosure, in the magnetic resonance imaging apparatus according to any one of the first aspect to the fourth aspect, it is preferable that the processor is configured to: acquire a margin designated by the user; and set the velocity encoding value for the main scan based on the margin and the maximum blood velocity.

According to a sixth aspect of the present disclosure, in the magnetic resonance imaging apparatus according to any one of the first aspect to the fifth aspect, it is preferable that the type of the target blood vessel includes at least one of an artery, a vein, an aorta, or a pulmonary artery.

According to a seventh aspect of the present disclosure, in the magnetic resonance imaging apparatus according to any one of the first aspect to the sixth aspect, it is preferable that the type of the target blood vessel includes at least one of an aorta during diastole or an aorta during systole.

According to an eighth aspect of the present disclosure, in the magnetic resonance imaging apparatus according to any one of the first aspect to the seventh aspect, it is preferable that the processor is configured to: display types of a plurality of blood vessels on a display device; and cause the user to select the type of the target blood vessel from among the displayed types of the plurality of blood vessels.

According to a ninth aspect of the present disclosure, in the magnetic resonance imaging apparatus according to any one of the first aspect to the eighth aspect, it is preferable that the image showing the blood velocity in the target blood vessel region is an absolute value image obtained by combining images showing velocities in each of three axial directions intersecting with each other.

According to a tenth aspect of the present disclosure, in the magnetic resonance imaging apparatus according to any one of the first aspect to the ninth aspect, it is preferable that the image showing the blood velocity in the target blood vessel region is a phase image showing a velocity in at least one axial direction of three axial directions intersecting with each other.

According to an eleventh aspect of the present disclosure, in the magnetic resonance imaging apparatus according to any one of the first aspect to the tenth aspect, it is preferable that the processor is configured to execute the pre-scan by applying a velocity encoding having a value for a pre-scan.

In order to achieve the above object, a method for operating a magnetic resonance imaging apparatus according to a twelfth aspect of the present disclosure is a method for operating a magnetic resonance imaging apparatus that measures a blood velocity of a blood vessel of a subject by applying a velocity encoding having a set value, the method comprising: via a processor, causing a user to input a type of a target blood vessel, which is the blood vessel to be measured, before a measurement start operation by the user; executing a pre-scan of the subject after the measurement start operation; detecting a target blood vessel region, which is a region of the target blood vessel, from an image obtained in the pre-scan; measuring a maximum blood velocity in the target blood vessel region from the image obtained in the pre-scan; setting a velocity encoding value for a main scan based on the maximum blood velocity; executing the main scan of the subject by applying the velocity encoding having the set value; and outputting an image showing the blood velocity in the target blood vessel region obtained in the main scan.

For the method for operating a magnetic resonance imaging apparatus according to the twelfth aspect, a configuration can be employed in which the same specific aspect as the magnetic resonance imaging apparatus according to any one of the first aspect to eleventh aspect.

In order to achieve the above object, a program according to a thirteenth aspect of the present disclosure is a program causing a computer to execute the method for operating a magnetic resonance imaging apparatus according to the twelfth aspect. The present disclosure also includes a non-transitory, tangible computer-readable storage medium having the program according to the thirteenth aspect stored therein.

According to the present invention, it is possible to measure a blood velocity by applying a velocity encoding having an appropriate value according to a type of a blood vessel designated by a user.

### BRIEF DESCRIPTION OF THE DRAWINGS

FIG. 1 is a diagram showing a schematic configuration of a magnetic resonance imaging apparatus.

FIG. 2 is a functional block diagram of a processor of a control device.

FIG. 3 is a flowchart showing steps of a blood flow measurement method that is an example of a method for operating the magnetic resonance imaging apparatus.

FIGS. 4A and 4B are diagrams for describing determination of an artery and a vein.

FIGS. 5A and 5B are diagrams for describing determination of the artery and the vein.

FIG. 6 is a diagram for describing determination of a systole and a diastole of the artery.

FIG. 7 is a diagram for describing extraction of a region of an aorta.

FIGS. 8A and 8B are diagrams showing an example of a UI screen for setting an item related to measurement of a blood velocity by a user.

FIGS. 9A to 9D are diagrams showing an example of an image of velocity data output after completion of a main scan for a target blood vessel.

### DESCRIPTION OF THE PREFERRED EMBODIMENTS

Hereinafter, a detailed description of preferred embodiments of the present invention will be made with reference to the accompanying drawings. In the present specification, the same reference numerals are assigned to the same components, and duplicate descriptions are omitted as appropriate.

### Configuration of Magnetic Resonance Imaging Apparatus

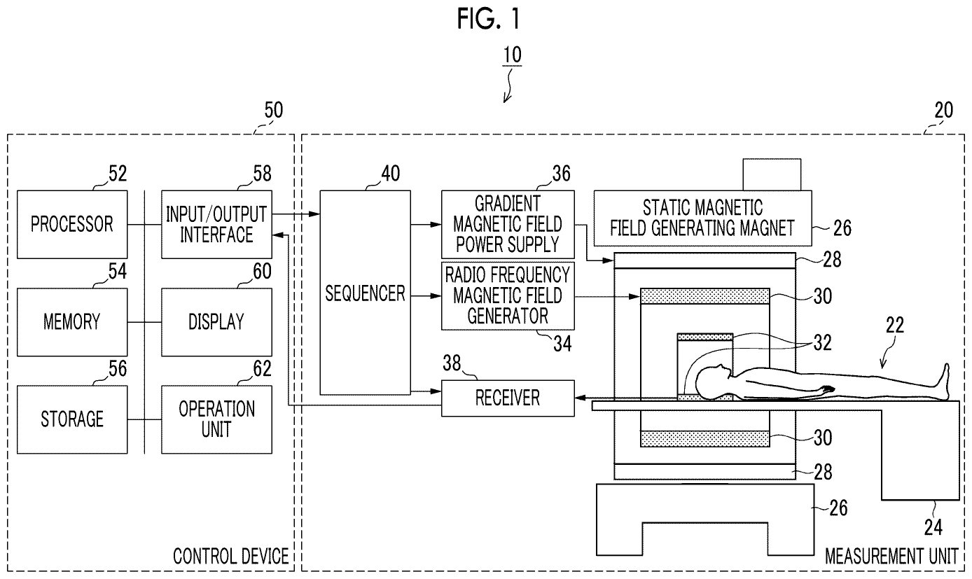

FIG. 1 is a diagram showing a schematic configuration of a magnetic resonance imaging apparatus. An MRI apparatus 10 shown in FIG. 1 comprises a measurement unit 20 and a control device 50. The measurement unit 20 comprises a static magnetic field generating magnet 26, a gradient magnetic field coil 28, a radio frequency (RF) coil 30, a receive coil 32, a high-frequency magnetic field generator 34, a gradient magnetic field power supply 36, a receiver 38, and a sequencer 40.

The measurement unit 20 includes a gantry having a cylindrical imaging space called a bore, and elements such as the static magnetic field generating magnet 26, the gradient magnetic field coil 28, and the RF coil 30 are disposed in the gantry. A subject 22 is usually disposed in the imaging space in the gantry in a state of lying on an examination table 24.

The static magnetic field generating magnet 26 generates a uniform static magnetic field in the imaging space in which the subject 22 is disposed. The static magnetic field generating magnet 26 includes a static magnetic field generating source of a permanent magnet type, a normal conducting type, or a superconducting type. The gradient magnetic field coil 28 generates a gradient magnetic field in the imaging space. The RF coil 30 generates a high-frequency magnetic field to induce a nuclear magnetic resonance (NMR) signal in atomic nuclei of atoms constituting tissues of the subject 22. The receive coil 32 detects the NMR signal generated from the subject 22.

The sequencer 40 transmits control information to the high-frequency magnetic field generator 34 and the gradient magnetic field power supply 36 in accordance with a pulse sequence (imaging sequence). The high-frequency magnetic field generator 34 generates a high-frequency current pulse of a Larmor frequency to induce nuclear magnetic resonance based on the control information input from the sequencer 40, and transmits the generated high-frequency current pulse to the RF coil 30. An RF pulse corresponding to the high-frequency current pulse is transmitted from the RF coil 30 to the subject 22.

The receive coil 32 detects the NMR signal generated by exciting the atomic nucleus spin in the subject 22 with the RF pulse. The NMR signal is usually collected as a gradient echo or a spin echo, and here, it is referred to as an echo signal. The receive coil 32 shown in FIG. 1 is an example of a coil that receives the echo signal from the head of the subject 22, but is not limited to the coil that receives the echo signal from the head, and a receive coil corresponding to an examination site such as a chest, an abdomen, a waist, a shoulder, or a hand or foot of the subject 22 is used and appropriately set. In addition, the receive coil 32 may be a flexible blanket type that is placed over the chest, the abdomen, and the waist.

The gradient magnetic field coil 28 is composed of an X-axis gradient magnetic field coil, a Y-axis gradient magnetic field coil, and a Z-axis gradient magnetic field coil that generate gradient magnetic fields Gx, Gy, and Gz in the X-axis direction, the Y-axis direction, and the Z-axis direction of the imaging space, respectively, and generates a gradient magnetic field corresponding to a current supplied from the gradient magnetic field power supply 36 in the imaging space. In a coordinate system of three orthogonal axes of the imaging space, a direction of a central axis of the gantry is usually defined as a Z-axis direction, a vertical direction is usually defined as a Y-axis direction, and a direction orthogonal to each of the Z-axis and the Y-axis is usually defined as an X-axis direction. The X-axis direction, the Y-axis direction, and the Z-axis direction are examples of “each axis direction of three axis directions intersecting with each other”, and the intersection is an example of “orthogonality”.

The Z-axis gradient magnetic field coil generates the gradient magnetic field Gz for selecting a slice position and a slice width of a plane of the subject 22 orthogonal to the Z-axis direction. The X-axis gradient magnetic field coil generates the gradient magnetic field Gx proportional to a position in the X-axis direction as a gradient magnetic field in a read-out direction (frequency-encoding direction) during a period in which an echo signal is generated. The Y-axis gradient magnetic field coil generates the phase-encoding direction gradient magnetic field Gy having different intensities for each repetition time TR.

The sequencer 40 controls each part to operate at a timing and intensity programmed in advance. In particular, a program that describes timings and intensity of the RF pulse, the gradient magnetic fields Gx, Gy, and Gz, and signal reception is referred to as a pulse sequence.

Various pulse sequences are known depending on the purpose, and examples thereof include a spin echo method and a gradient echo method, and there are various pulse sequences derived from these.

The NMR signal generated from the subject 22 is detected by the receive coil 32, is amplified by a preamplifier (not shown) in the receive coil 32, and is transmitted to the receiver 38.

The receiver 38 includes a quadrature detection circuit, an analog to digital (A/D) converter, and other signal processing circuits. In the receiver 38, the amplified NMR signal is subjected to A/D conversion and necessary signal processing to generate data. The data generated in this way is transmitted to the control device 50.

For example, the receiver 38 executes detection and A/D conversion of the echo signal detected by the receive coil 32, and transmits two series of digital data of a real part and an imaginary part, which are k-space data sequentially collected along a k-trajectory from the echo signal, to the control device 50. The digital data is also referred to as measurement data.

The sequencer 40 transmits information on a nuclear magnetic resonance frequency (detection reference frequency) that is a reference for quadrature detection performed in the receiver 38, a timing of the A/D conversion (sampling timing in the frequency-encoding direction), and the like, and controls the receiver 38.

A reception-side cable (not shown) that outputs the NMR signal received by the receive coil 32 is connected to the receive coil 32. Although not shown in FIG. 1, a reception-side connector is connected to an end portion of the reception-side cable. The reception-side connector is connected to an examination table-side connector provided on the examination table 24. The examination table-side connector is connected to an examination table-side cable disposed inside the examination table 24, and the examination table-side cable is connected to the control device 50.

As a result, the receive coil 32, the control device 50, and the sequencer 40 are communicably connected to each other. The connection between the receive coil 32, and the control device 50 and the sequencer 40 is not limited to wired connection, such as through a cable, and can also be established wirelessly. In this case, the receive coil 32 or the examination table 24 further includes at least an A/D conversion module and a wireless communication module.

The control device 50 is an example of an image processing apparatus that causes a processor to image data acquired by imaging a subject. The control device 50 controls the measurement unit 20 and performs various types of operations such as image reconstruction based on the signal obtained from the measurement unit 20. The control device 50 can be configured by using a computer. The computer applied to the control device 50 may be a personal computer, a workstation, or a server computer.

The control device 50 includes, as hardware, a processor 52, a memory 54, a storage 56, an input/output interface 58, a display 60, and an operation unit 62.

The processor 52 includes a central processing unit (CPU). The processor 52 may include a graphics processing unit (GPU). In addition, the processor 52 may include one or a plurality of pieces of hardware of a digital signal processor (DSP), an application-specific integrated circuit (ASIC), a programmable logic device (PLD), and the like.

The processor 52 performs overall control of each unit of the control device 50 and the measurement unit 20, and executes various programs stored in the memory 54 to realize various functions.

The memory 54 is a storage device including a random access memory (RAM). The memory 54 may include one or more of a flash memory and a read-only memory (ROM). The storage 56 is a storage device including, for example, one or more of a hard disk apparatus, a solid-state drive apparatus, and a removable recording medium. The flash memory, the ROM, and the storage 56 are non-volatile storage devices that store an operation system, a program for causing the processor 52 to function as the control device 50, various pulse sequences, calculation expressions used for image reconstruction, parameters, trained models, MR images subjected to image reconstruction, and the like.

The RAM functions as a work area for processing by the processor 52, and temporarily stores programs and the like stored in the non-volatile storage device. In addition, the RAM functions as a place for temporarily storing measurement data, which is digital sampling data obtained by processing the echo signal obtained from the measurement unit 20, and so-called raw data. It should be noted that a part (RAM) of the memory 54 may be built into the processor 52.

The input/output interface 58 includes a communication unit that can be connected to a network, a connection unit that can be connected to an external apparatus, and the like. As the connection unit that can be connected to the external apparatus, a universal serial bus (USB) (USB is a registered trademark), a high-definition multimedia interface (HDMI) (HDMI is a registered trademark), or the like can be applied.

The processor 52 communicates with the receiver 38 and the sequencer 40 of the measurement unit 20 via the input/output interface 58 to transmit and receive necessary information. A part of the sequencer 40 may be provided on the control device 50 side.

The display 60 is, for example, a display device configured by using a liquid crystal display, an organic electro-luminescence (OEL) display, a projector, or an appropriate combination thereof. Various types of information are displayed on the display 60 in addition to the MRI image captured by the MRI apparatus 10. The display 60 is used as a part of a user interface (UI) in a case of receiving an input from the operation unit 62. The display 60 is not limited to one, and a multi-display form comprising a plurality of display devices is also possible.

The operation unit 62 includes an input device such as a mouse and a keyboard, and functions as a part of a graphical user interface (GUI) that receives an input from a user such as a radiologist using a display operation window of the display 60. For example, the display 60 and the operation unit 62 function as a GUI for the user to input the start and stop (temporary stop) of the measurement unit 20, the selection of the pulse sequence, the imaging conditions, the processing conditions, and the like. The operation unit 62 may include a voice input device. In addition, the operation unit 62 may be a touch panel type input device configured integrally with a display screen of the display 60.

### Functional Configuration of Processor

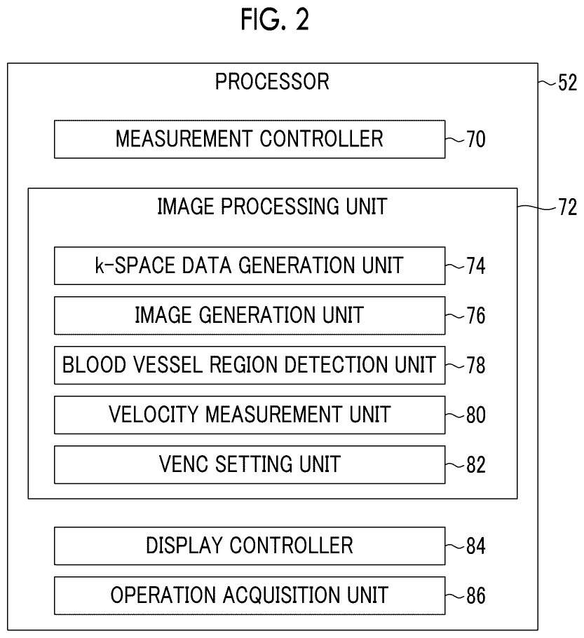

FIG. 2 is a functional block diagram of the processor 52 of the control device 50. The processor 52 executes various programs stored in the memory 54 to function as a measurement controller 70, an image processing unit 72, a display controller 84, and an operation acquisition unit 86.

The measurement controller 70 sends a predetermined pulse sequence, user-specified imaging conditions (imaging parameters), and the like to the sequencer 40 of the measurement unit 20, and controls the measurement unit 20 via the sequencer 40. In the present embodiment, a known PC method pulse sequence in which a pair of velocity encode gradient magnetic field pulses are applied after the application of the excitation RF pulse is used.

The measurement controller 70 sends a command to the sequencer 40 in accordance with the set conditions, and causes the measurement unit 20 to execute the pulse sequence under the control of the sequencer 40. The subject 22 is imaged by executing the pulse sequence by the measurement unit 20, and the receive coil 32 provided at the examination site of the subject 22 receives the echo signal from the subject 22. The receiver 38 executes amplification, detection, and A/D conversion of the echo signal received by the receive coil 32, and transmits digital data of a real part and an imaginary part to the control device 50 via the input/output interface 58.

The image processing unit 72 includes a k-space data generation unit 74, an image generation unit 76, a blood vessel region detection unit 78, a velocity measurement unit 80, a VENC setting unit 82, and the like.

The k-space data generation unit 74 arranges the k-space data, which is the digital data of the real part and the imaginary part and is sequentially collected from the echo signal along the k-trajectory, in the k-space. In a case in which the size of a field of view (FOV) is 256pixels×256 pixels, the entire region of the k-space also has a size of 256×256.

The image generation unit 76 performs inverse Fourier transform (inverse fast Fourier transform (FFT)) on the k-space data generated by the k-space data generation unit 74 to generate a reconstructed image (MR image) in a real space. The MR image obtained by the pulse sequence of the PC method may be an image in which a velocity vector value of blood is a pixel value.

The blood vessel region detection unit 78 detects a blood vessel region from the MR image generated by the image generation unit 76. The blood vessel region detection unit 78 may determine an artery and a vein from the variation in the blood velocity, or may detect a blood vessel region by image analysis.

The velocity measurement unit 80 measures the velocity (flow velocity) of blood in the blood vessel region detected by the blood vessel region detection unit 78 from the MR image generated by the image generation unit 76. The velocity measurement unit 80 may measure the maximum velocity of blood.

The VENC setting unit 82 calculates and sets a VENC value required for imaging of the PC method based on the maximum velocity measured by the velocity measurement unit 80. The VENC setting unit 82 may calculate the VENC value including a margin. The measurement controller 70 applies velocity encoding of the set VENC value and performs a main scan described below.

The display controller 84 displays the MR image calculated by the image processing unit 72 and accessory information such as imaging conditions and information related to the subject 22 on the display 60, and displays the MR image and the like recorded in the memory 54 on the display 60.

The operation acquisition unit 86 acquires the activation, the stop, the selection of the pulse sequence, the imaging conditions, the processing conditions, and the like of the measurement unit 20 input by the user using the operation unit 62.

Blood Flow Measurement Method

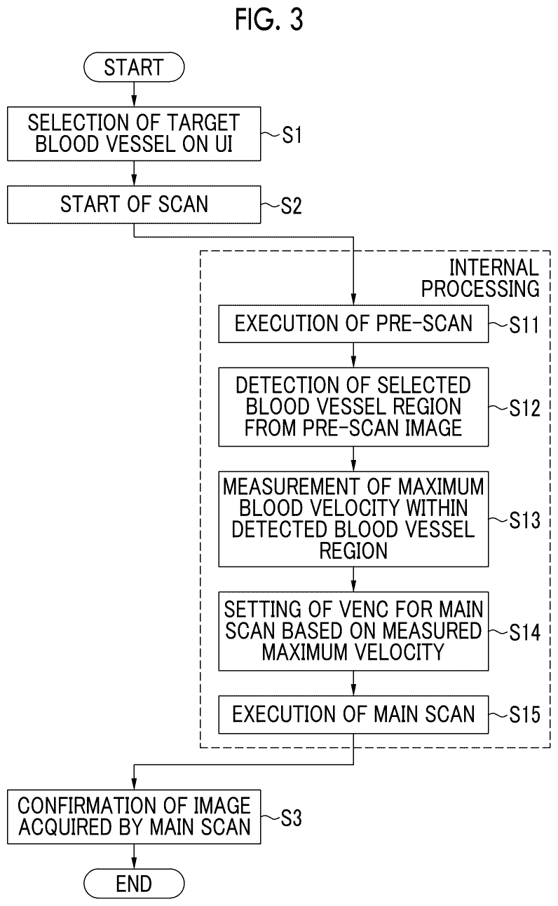

FIG. 3 is a flowchart showing steps of a blood flow measurement method that is an example of a method for operating a magnetic resonance imaging apparatus. Here, an example of measuring a blood flow by the PC method using the MRI apparatus 10 will be described. In FIG. 3, steps S1 to S3 are operations or actions of the user, and steps S11 to S15 are internal processing of the processor 52 of the control device 50. The processing of the processor 52 is realized by the processor 52 executing an operation program of the magnetic resonance imaging apparatus stored in the memory 54.

First, the user places the subject 22 on the examination table 24, disposes the examination table 24 on which the subject 22 is placed in the imaging space in the gantry, and brings the examination site of the subject 22 into a state in which the examination site can be imaged.

In subsequent step S1, the user selects (an example of “input”) a target blood vessel, which is a blood vessel as a measurement target, on the user interface before the imaging of the subject 22. For example, the user selects the type of the target blood vessel from among the types of a plurality of blood vessels displayed on the display 60 by using the operation unit 62. The display controller 84 may display the types of the plurality of blood vessels on the display 60, and the operation acquisition unit 86 may acquire the type of the target blood vessel selected by the user from among the displayed types of the plurality of blood vessels. The user may select a plurality of target blood vessels. The user may designate the type of the target blood vessel by directly inputting the name of the type of the blood vessel by using the operation unit 62.

In addition, in step S1, the user may designate a margin in a case of calculating the VENC value. The margin here is a degree of a margin of the VENC value with respect to the maximum blood velocity. In a case in which a plurality of target blood vessels are selected, the user may designate a margin of each target blood vessel. The operation acquisition unit 86 may acquire the designated margin in association with the target blood vessel.

In step S2, the user causes the MRI apparatus 10 to start imaging (scanning) of the subject 22. For example, the user starts the measurement of the blood flow of the target blood vessel by operating a UI button for starting the measurement displayed on the display 60 by using the operation unit 62 (an example of a “measurement start operation”). The display controller 84 may display the UI button on the display 60, and the operation acquisition unit 86 may acquire the operation of the displayed UI button by the user. As described above, the processor 52 causes the user to input the type of the target blood vessel, and then receives the measurement start operation by the user.

In step S11, the measurement controller 70 causes the measurement unit 20 to execute the pre-scan based on the type of the blood vessel selected in step S1 and the operation of the UI button input in step S2. As described above, the processor 52 executes the pre-scan of the subject 22 after the measurement start operation by the user.

The pre-scan is a scan having a relatively lower spatial resolution or temporal resolution than a main scan described below. In the pre-scan, an approximate blood velocity in the blood vessel region is measured in order to set an appropriate VENC value during the main scan. In the pre-scan, a pulse sequence of a known two-dimensional PC method (2D-PC method) or three-dimensional PC method (3D-PC method) in which a pair of velocity encoding gradient magnetic field pulses for encoding the velocity after the application of the excitation RF pulse is applied is used. The pair of velocity encoding gradient magnetic field pulses is applied with an intensity corresponding to the VENC value. The VENC value in the pre-scan may be a value set in advance for the pre-scan. The VENC value in the pre-scan may be a sufficiently large value. The pre-scan may be executed for the entire cardiac phase.

Further, in step S11, the k-space data generation unit 74 arranges the k-space data sequentially collected along the k-trajectory from the acquired echo signal in the k-space. Subsequently, the image generation unit 76 generates a pre-scan image, which is an MR image, from the k-space data generated by the k-space data generation unit 74.

In step S12, the blood vessel region detection unit 78 detects a target blood vessel region, which is a region of the target blood vessel selected in step S1, from the pre-scan image generated in step S11. The blood vessel region detection unit 78 may detect only the region of the target blood vessel selected in step S1. In a case in which a plurality of target blood vessels are selected in step S1, the blood vessel region detection unit 78 detects each target blood vessel region from the pre-scan image.

In step S13, the velocity measurement unit 80 measures a maximum blood velocity in the target blood vessel region detected in step S12 from the pre-scan image generated in step S11. In a case in which a plurality of target blood vessels are selected in step S1, the velocity measurement unit 80 measures the maximum blood velocity in each target blood vessel region.

In step S14, the VENC setting unit 82 calculates and sets a VENC value for the main scan based on the maximum velocity measured in step S13. In a case in which the maximum velocity measured in step S13 is denoted by Vmax1 (unit: centimeters per second) and the margin designated in step S1 is denoted by M1 (unit: percent) for the first target blood vessel, a VENC value1, which is the VENC value of the first target blood vessel, is calculated by the following Equation 1.

VENC value1=Vmax1×(1+M1/100) ... (Equation 1)

In a case in which a plurality of target blood vessels are selected in step S1, the VENC setting unit 82 sets the VENC value of each target blood vessel. For example, in a case in which a second target blood vessel different from the first target blood vessel is selected, a VENC value2, which is the VENC value of the second target blood vessel, is calculated by the following Equation 2 in a case in which the maximum velocity measured in step S13 is denoted by Vmax2 (unit: centimeters per second) and the margin designated in step S1 is denoted by M2 (unit: percent) for the second target blood vessel.

VENC value2=Vmax2×(1+M2/100) ... (Equation 2)

Subsequently, in step S15, the measurement controller 70 causes the measurement unit 20 to execute the main scan. In the main scan, a pulse sequence of a known two-dimensional PC method or three-dimensional PC method in which a pair of velocity encoding gradient magnetic field pulses are applied after the application of the excitation RF pulse is used. The pair of velocity encoding gradient magnetic field pulses is applied with an intensity corresponding to the VENC value. The VENC value in the main scan is set to the value set in step S14. The main scan may be executed for the entire cardiac phase. In a case in which a plurality of target blood vessels are selected in step S1, the measurement controller 70 executes the main scan a number of times corresponding to the number of types of VENC values. The relationship between the type of the VENC value and the order of the main scan is not particularly limited.

In addition, in step S15, the k-space data generation unit 74 generates the k-space data from the echo signal acquired by the main scan. The image generation unit 76 generates the main scan image, which is the MR image, from the k-space data generated by the k-space data generation unit 74. The main scan image may be an image showing the blood velocity data of the target blood vessel region. The display controller 84 displays the main scan image on the display 60. In a case in which a plurality of target blood vessels are selected in step S1, the display controller 84 may display the main scan image of each target blood vessel on the display 60.

Finally, in step S3, the user checks the main scan image displayed on the display 60. As a result, the user can recognize the blood velocity of the selected target blood vessel.

In the technique disclosed in JP2000-175885A, in a case in which “artery” is selected, a VENC value is set to acquire the blood velocity of not only the artery but also the “vein” to eliminate the vein. On the other hand, the blood flow measurement method according to the present embodiment measures only the maximum velocity of the target blood vessel region selected by the user from the pre-scan image. For example, in a case in which the user selects “artery”, the VENC value is set without considering blood vessels other than the artery. As a result, according to the blood flow measurement method according to the present embodiment, it is possible to output an image of the velocity data focusing on the target blood vessel selected by the user.

### Method 1 for Detecting Blood Vessel Region From Pre-Scan Image

The processor 52 may determine whether or not the blood vessel region is the target blood vessel region based on the variation in the blood velocity in the time direction. For example, it is possible to determine whether the blood vessel is an artery or a vein from the variation in the blood velocity in the time direction. The variation in the blood velocity in the time direction may be a difference between a time phase at which the blood velocity is maximum and a time phase at which the blood velocity is minimum.

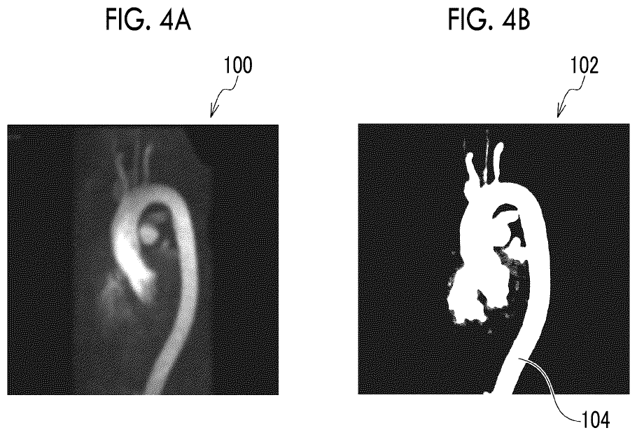

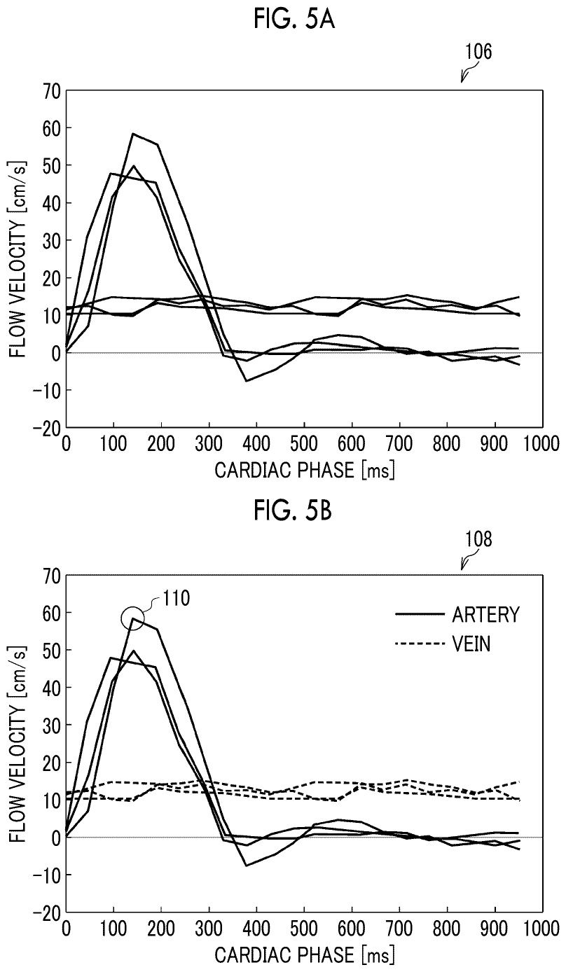

FIGS. 4A to 5B are diagrams for describing the determination of the artery and the vein. An image 100 of FIG. 4A is an example of the pre-scan image generated in step S11. The image 100 is acquired in each entire cardiac phase. In addition, an image 102 of FIG. 4B is a mask image created from the image 100. The image 102 is created, for example, by the image processing unit 72 performing binarization processing on the image 100. As a result, in the image 102, the blood vessel region is represented as a white mask region 104.

A graph 106 of FIG. 5A is a graph in which the blood velocity for each pixel in a region of the image 100 corresponding to the mask region 104 of the image 102 is plotted. A horizontal axis of the graph 106 indicates a cardiac phase (unit: milliseconds), and a vertical axis indicates a flow velocity (unit: centimeters per second).

The processor 52 calculates a variation in the velocity in the time direction for each pixel from the graph 106, and determines whether the pixel is in the artery region or the vein region. For example, standard deviations σp of the changes over time in the blood velocity of a certain pixel p are as follows.
σ1=1, σ2=1.1, σ3=10, σ4=10.1, σ5=1.2, σ6=11, ...

The processor 52 divides the standard deviations σp into two groups as follows by a k-means method or the like.
Small variation=[σ1=1, σ2=1.1, σ5=1.2, ...]
   Large variation=[σ3=10, σ4=10.1, σ6=11, ...]

Then, the processor 52 determines that pixels p1, p2, p5, ... having a small variation are in the vein region, and determines that pixels p3, p4, p6, ... having a large variation are in the artery region. Further, the processor 52 measures the maximum blood velocity in the target blood vessel region. For example, in a case where the target blood vessel is an artery, the maximum blood velocity in the region determined to be the artery region is measured.

A graph 108 in FIG. 5B is the same graph as the graph 106, and a plot position 110 indicating the maximum velocity of the artery region is shown. The processor 52 may measure the velocity at the plot position 110 and set the VENC value.

In addition, the target blood vessel may be the same blood vessel, and a difference in state may be designated. For example, the user may be able to designate not only the aorta but also any of the diastole or the systole.

In this case, the processor 52 determines the region of the artery from the variation in the velocity in the time direction. Subsequently, the processor 52 further determines whether the determined region of the artery is the systole or the diastole. Here, the cardiac phase is acquired by an electrocardiogram (ECG) sensor or a pulse wave sensor during the pre-scanning, and the pre-scanning image and the imaging time point are associated with each other.

For example, in a case where a velocity at a time point t within a cardiac cycle of a certain pixel p is denoted by vp,t, it is assumed that the velocities vp,t for the pixel 1 (where p=1) are as follows.
v1,1=30, v1,2=40, v1,3=20, v1,4=5, v1,5=3, v1,6=4, ...

The processor 52 divides the velocities vp,t into two groups as follows by a k-means method or the like.
High velocity=[v1,1=30, v1,2=40, v1,3=20, ...]
   Low velocity=[v1,4=5, v1,5=3, v1,6=4, ...]

Then, the processor 52 determines that the time points t=1, 2, 3, ... of the high velocity are the systole and the time points t=4, 5, 6, ... of the low velocity are the diastole.

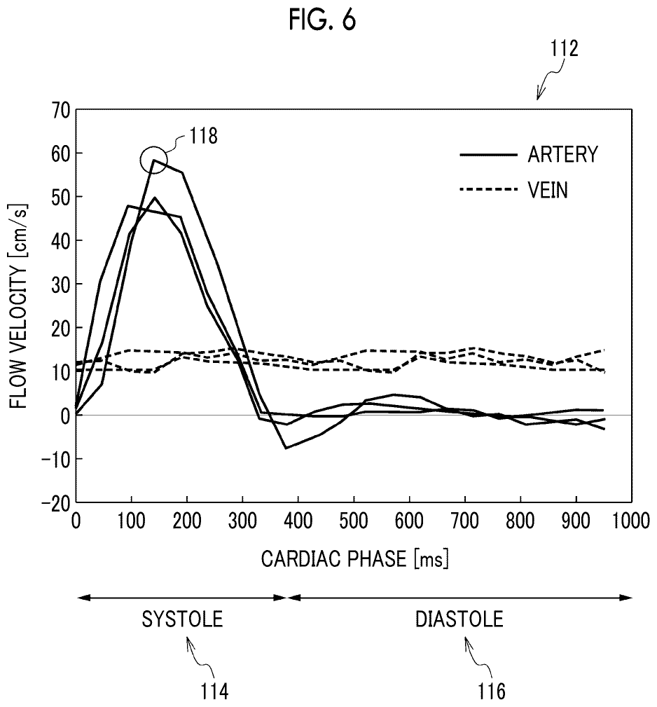

FIG. 6 is a diagram for describing determination of the systole and the diastole of the artery. A graph 112 of FIG. 6 is the same graph as the graph 106 of FIG. 5A, and shows a determined systole 114 and a determined diastole 116.

The processor 52 measures the maximum velocity of the blood in the target blood vessel region. For example, in a case in which the target blood vessel is the systole of the artery, the maximum velocity in the time phase of the systole is measured for all pixels in the region determined as the artery region.

The graph 112 of FIG. 6 shows a plot position 118 indicating the maximum velocity of the systole of the artery. The processor 52 may measure the velocity at the plot position 118 and set the VENC value.

### Method 2 for Detecting Blood Vessel Region from Pre-Scan Image

The processor 52 may generate a three-dimensional image of the blood vessel from the pre-scan image and automatically extract the target blood vessel region by a known automatic analysis technique. Here, a case in which the target blood vessel is the aorta will be described.

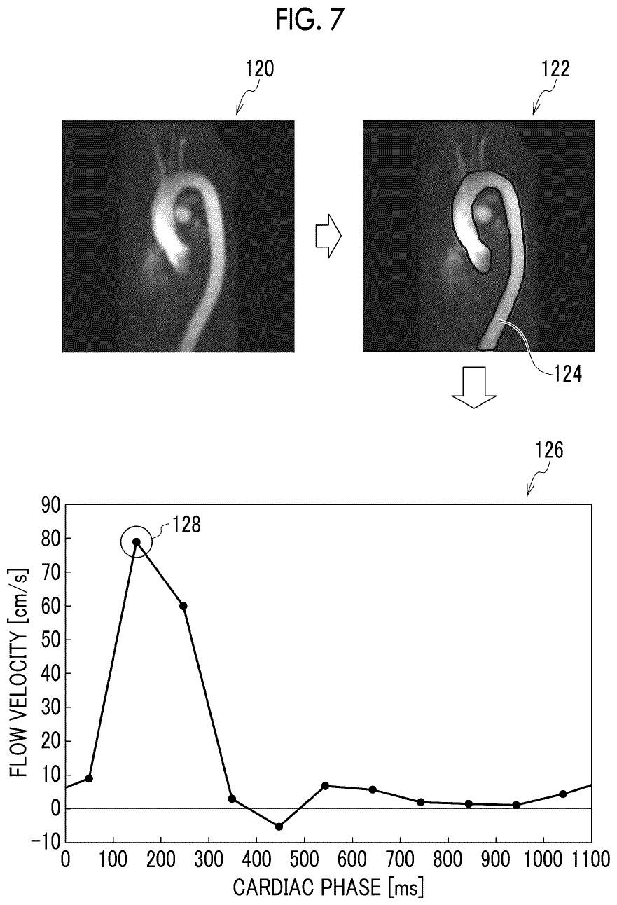

FIG. 7 is a diagram for describing extraction of a region of the aorta. An image 120 of FIG. 7 is an example of the pre-scan image acquired in step S11. In addition, an image 122 of FIG. 7 is an image in which a region 124 of the aorta extracted from the image 120 is surrounded by a solid line and displayed in an emphasized manner. The processor 52 generates a 3D image of the blood vessel from the image 120, extracts the region of the aorta from the 3D image by an image analysis technique, and extracts the region 124 of the aorta in the image 120. The processor 52 may extract the region 124 of the aorta from the image 120 by an image analysis technique.

The processor 52 measures the maximum blood velocity in the extracted region 124 of the aorta. Here, the maximum blood velocity in the entire cardiac phase is measured for all pixels in the region 124 determined as the aortic region.

A graph 126 of FIG. 7 is a graph in which the blood velocity in the entire cardiac phase in the region 124 is plotted. A horizontal axis of the graph 126 indicates a cardiac phase (unit: milliseconds), and a vertical axis indicates a flow velocity (unit: centimeters per second). A plot position 128 indicating the maximum blood velocity is shown in the graph 126. The processor 52 may measure the blood velocity at the plot position 128 and set the VENC value.

UI Screen

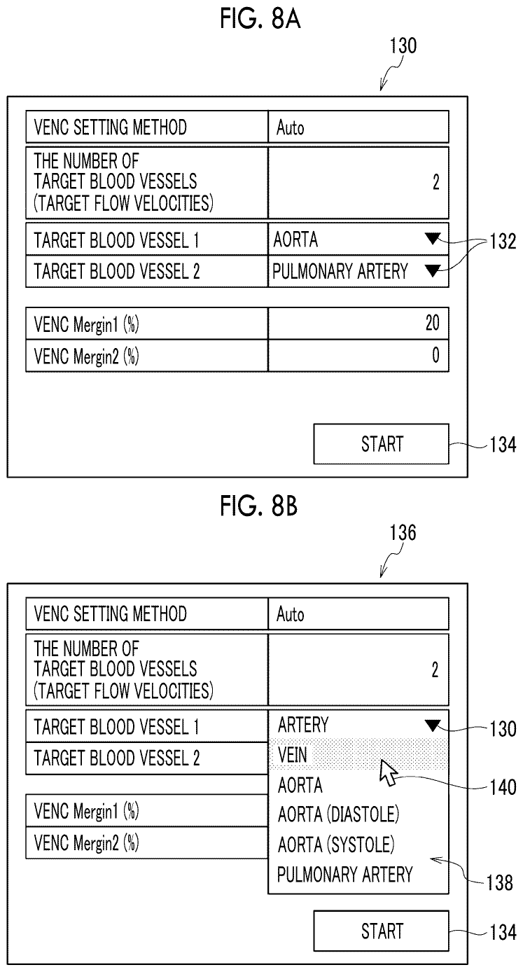

FIGS. 8A and 8B are diagrams showing an example of a UI screen for the user to designate items related to the blood flow measurement. A UI screen 130 shown in FIG. 8A is displayed on the display 60 in, for example, step S1 of FIG. 3. The UI screen 130 includes, as designated items, a “VENC setting method”, the “number of target blood vessels (target flow velocities)”, a “target blood vessel 1”, a “target blood vessel 2”, a “VENC Mergin1 (%)”, and a “VENC Mergin2 (%)”.

The “VENC setting method” is an item for designating whether to automatically set the VENC value or to manually set the VENC value by the user. In the UI screen 130, the “VENC setting method” is designated as “Auto” in which the VENC value is automatically set. The “VENC setting method” may be designated as “Manual” in which the user manually sets the VENC value. In this case, the user designates the VENC value on a screen different from the UI screen 130, but the description thereof will be omitted here.

The “number of target blood vessels (target flow velocities)” is an item for designating the number of types of target blood vessels for which the blood velocity is to be measured. The user can designate any natural number as the “number of target blood vessels (target flow velocities)”. For example, the user designates “1” in a case of designating only the aorta, designates “2” in a case of designating the aorta and the pulmonary artery, and designates “3” in a case of measuring the aorta, the pulmonary artery, and the vein, as the “number of target blood vessels (target flow velocities)”. In the UI screen 130, the “number of target blood vessels (target flow velocities)” is designated as “2” indicating that the number of types of target blood vessels to be measured is two.

The “target blood vessel 1” and the “target blood vessel 2” are items for designating the type of the target blood vessel for which the blood velocity is to be measured. In the UI screen 130, since the “number of target blood vessels (target flow velocities)” is set to “2”, the “target blood vessel 1” and the “target blood vessel 2” are displayed. In a case where the “number of target blood vessels (target flow velocities)” is set to “1”, only the “target blood vessel 1” is displayed as the item for designating the type of the target blood vessel. In a case where the “number of target blood vessels (target flow velocities)” is set to “3”, in addition to the “target blood vessel 1” and the “target blood vessel 2”, the “target blood vessel 3” is further displayed.

In the UI screen 130, the “aorta” is designated as the “target blood vessel 1”, and the “pulmonary artery” is designated as the “target blood vessel 2”.

The “target blood vessel 1” and the “target blood vessel 2” include downward-pointing triangular icons 132. In a case where the icon 132 is selected, a pull-down menu is displayed. A UI screen 136 shown in FIG. 8B is a state in which the icon 132 of the “target blood vessel 1” is selected on the UI screen 130, and a pull-down menu 138 is displayed. The user may operate a mouse (not shown) of the operation unit 62 to move a cursor 140 to a position of the icon 132 of the “target blood vessel 1” and perform a click operation to select the icon 132 of the “target blood vessel 1”.

A plurality of types of blood vessels are displayed in the pull-down menu 138 to be selectable by the user. Here, the pull-down menu 138 includes “artery”, “vein”, “aorta”, “aorta (diastole)”, “aorta (systole)”, and “pulmonary artery” as the types of blood vessels.

In order to select the “target blood vessel 1” from among the plurality of types of blood vessels displayed in the pull-down menu 138, for example, the user may operate the mouse to move the cursor 140 to a position of a desired type of blood vessel in the pull-down menu 138 and perform a click operation.

In a case where any type of blood vessel is selected from among the plurality of types of blood vessels displayed in the pull-down menu 138, the display of the pull-down menu 138 disappears, and the selected type of blood vessel is displayed as the “target blood vessel 1”. As a result, the selected type of blood vessel is designated as the “target blood vessel 1”. The same applies to the designation of the “target blood vessel 2”.

“VENC Mergin1 (%)” and “VENC Mergin2 (%)” are items for setting a margin (unit: percent) in a case of setting the VENC value of the “target blood vessel 1” and the “target blood vessel 2”, respectively. On the UI screen 130, “20” is set as “VENC Mergin1 (%)” and “0” is set as “VENC Mergin2 (%)”. The “VENC Mergin1 (%)” and the “VENC Mergin2 (%)” may be displayed in advance with a value such as “0” or “20” as an initial value.

In addition, the UI screen 130 includes a start button 134. The start button 134 is a UI button for the user to start the measurement of the blood velocity of the blood vessel of the subject. In a case where the start button 134 is operated, the processor 52 starts the internal processing of steps S11 to S15 in FIG. 3. The operation of the start button 134 may be performed by the user operating the mouse to move the cursor 140 to the position of the start button 134 and performing a click operation. The start button 134 may be displayed in a grayed-out manner in an inactive state until all the items of the “method for setting VENC”, the “number of target blood vessels (target flow velocities)”, the “target blood vessel 1”, the “target blood vessel 2”, the “VENC Mergin1 (%)”, and the “VENC Mergin2 (%)” are designated.

### Output Image

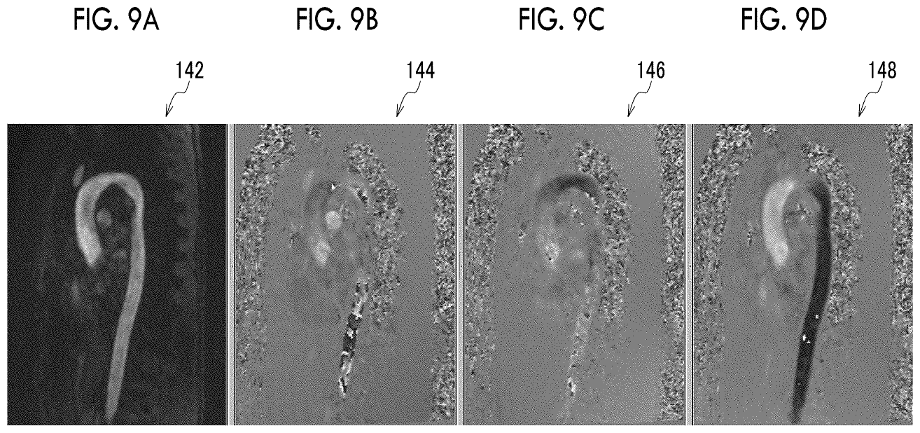

FIGS. 9A to 9D are diagrams showing an example of an image showing the blood velocity in the target blood vessel region obtained by the main scan. Each of FIGS. 9A to 9D shows an image acquired by executing the main scan with the velocity encoding in which the VENC value is set to 120 (unit: centimeters per second). An image 142 is an absolute value image and is a three-axis velocity composite image in which images showing the velocities in the X-axis direction, the Y-axis direction, and the Z-axis direction are combined. In the image 142, a tissue having a high velocity is represented by a relatively high signal, that is, relatively white.

Images 144, 146, and 148 are phase images and are an X-axis velocity image, a Y-axis velocity image, and a Z-axis velocity image showing the velocities in the X-axis direction, the Y-axis direction, and the Z-axis direction, respectively. In the images 144, 146, and 148, a tissue having a relatively high velocity in the positive direction is represented to be whiter, a tissue having a relatively high velocity in the negative direction is represented to be blacker, and a tissue having a velocity relatively close to 0 is represented to be intermediate gray.

The display controller 84 may display at least one of the images 142, 144, 146, or 148 on the display 60 after the main scan is completed.

### Processor

In the present embodiment, each type of processing is executed by any computer. In addition, any computer may execute these processes using a processor as hardware, a program as software, or a combination thereof. In such a case, the processor is configured to execute various types of processes in the present embodiment in cooperation with the program, and may function as each unit or each means in the present embodiment. Additionally, the execution order of the process by the processor is not limited to the order described above and may be changed as appropriate. Any computer may be a general purpose computer, a special purpose computer, a workstation, or other system capable of executing each process.

The processor may be configured with one or more pieces of hardware, and the type of hardware is not limited. For example, the processor may be configured by a programmable logic device such as a central processing unit (CPU), a micro processing unit (MPU), or a field programmable gate array (FPGA), a dedicated circuit for executing specific processing, such as an application-specific integrated circuit (ASIC), or hardware such as a graphics processing unit (GPU) or a neural processing unit (NPU). Additionally, the type of hardware may be a combination of different types of hardware. In a case in which the plurality of types of hardware are configured to execute one or a plurality of types of processing of a certain processor, the plurality of types of hardware may exist in devices physically separated from each other or may exist in the same device. In addition, in any of the embodiments, the order of the processes performed by the processor is not limited to the order described above, and may be changed as appropriate. The hardware is configured by an electric circuit (circuitry) in which circuit elements such as semiconductor elements are combined.

Further, the program may be software, such as firmware or a microcode. Furthermore, the program may be, for example, a program module group, and each function thereof may be implemented by a processor configured to execute each function. The program may be a program code or a plurality of code segments that are stored in one or a plurality of non-transitory computer-readable media (for example, storage media or other storages). The program may be divided and stored in a plurality of non-transitory computer-readable media present in devices physically separated from each other. The program code or the code segments may represent any combination of a procedure, a function, a subprogram, a routine, a subroutine, a module, a software package, a class, an instruction, a data structure, and a program statement. The program code or code segments may be connected to other code segments or to hardware circuits by transmitting and receiving information, data, arguments, parameters, or contents of memory.

### Other

In addition, the present invention can also be applied to a program and a program product.

The technical scope of the present invention is not limited to the scope described in the above-described embodiments. The configurations and the like in each embodiment can be appropriately combined between the respective embodiments without departing from the spirit of the present invention.

### EXPLANATION OF REFERENCES

10: MRI apparatus
20: measurement unit 22: subject
24: examination table
26: static magnetic field generating magnet
28: gradient magnetic field coil
30: RF coil
32: receive coil
34: high-frequency magnetic field generator
36: gradient magnetic field power supply
38: receiver
40: sequencer
50: control device
52: processor
54: memory
56: storage
58: input/output interface
60: display
62: operation unit
70: measurement controller
72: image processing unit
74: k-space data generation unit
76: image generation unit
78: blood vessel region detection unit
80: velocity measurement unit
82: VENC setting unit
84: display controller
86: operation acquisition unit
100: image
102: image
104: mask region
106: graph
108: graph
110: plot position
112: graph
114: systole
116: diastole
118: plot position
120: image
122: image
124: region
126: graph
128: plot position
130: UI screen
132: Icon
134: start button
136: UI screen
138: pull-down menu
140: cursor
142: image
144: image
146: image
S1 to S3, S11 to S15: step of blood flow measurement method

What is claimed is:

1. A magnetic resonance imaging apparatus that measures a blood velocity of a blood vessel of a subject by applying a velocity encoding having a set value, the magnetic resonance imaging apparatus comprising:
a processor,
wherein the processor is configured to:
cause a user to input a type of a target blood vessel, which is the blood vessel to be measured, before a measurement start operation by the user;
execute a pre-scan of the subject after the measurement start operation;
detect a target blood vessel region, which is a region of the target blood vessel, from an image obtained in the pre-scan;
measure a maximum blood velocity in the target blood vessel region from the image obtained in the pre-scan;
set a velocity encoding value for a main scan based on the maximum blood velocity;
execute the main scan of the subject by applying the velocity encoding having the set value; and
output an image showing the blood velocity in the target blood vessel region obtained in the main scan.

2. The magnetic resonance imaging apparatus according to claim 1,
wherein the processor is configured to:
detect a blood vessel region, which is a region of a blood vessel, from the image obtained in the pre-scan;
acquire a variation over time in blood velocity in the blood vessel region; and
determine whether or not the blood vessel region is the target blood vessel region based on the variation in the blood velocity.

3. The magnetic resonance imaging apparatus according to claim 1,
wherein the processor is configured to:
generate a three-dimensional image of a blood vessel from the image obtained in the pre-scan; and
analyze the three-dimensional image to extract the target blood vessel region.

4. The magnetic resonance imaging apparatus according to claim 1,
wherein the processor is configured to set the velocity encoding value for the main scan for each of a plurality of the target blood vessels.

5. The magnetic resonance imaging apparatus according to claim 1,
wherein the processor is configured to:
acquire a margin designated by the user; and
set the velocity encoding value for the main scan based on the margin and the maximum blood velocity.

6. The magnetic resonance imaging apparatus according to claim 1,
wherein the type of the target blood vessel includes at least one of an artery, a vein, an aorta, or a pulmonary artery.

7. The magnetic resonance imaging apparatus according to claim 1,
wherein the type of the target blood vessel includes at least one of an aorta during diastole or an aorta during systole.

8. The magnetic resonance imaging apparatus according to claim 1,
wherein the processor is configured to: display types of a plurality of blood vessels on a display device; and
cause the user to select the type of the target blood vessel from among the displayed types of the plurality of blood vessels.

9. The magnetic resonance imaging apparatus according to claim 1,
wherein the image showing the blood velocity in the target blood vessel region is an absolute value image obtained by combining images showing velocities in each of three axial directions intersecting with each other.

10. The magnetic resonance imaging apparatus according to claim 1,
wherein the image showing the blood velocity in the target blood vessel region is a phase image showing a velocity in at least one axial direction of three axial directions intersecting with each other.

11. The magnetic resonance imaging apparatus according to claim 1,
wherein the processor is configured to execute the pre-scan by applying a velocity encoding having a value for a pre-scan.

12. A method for operating a magnetic resonance imaging apparatus that measures a blood velocity of a blood vessel of a subject by applying a velocity encoding having a set value, the method comprising:
via a processor,
causing a user to input a type of a target blood vessel, which is the blood vessel to be measured, before a measurement start operation by the user;
executing a pre-scan of the subject after the measurement start operation;
detecting a target blood vessel region, which is a region of the target blood vessel, from an image obtained in the pre-scan;
measuring a maximum blood velocity in the target blood vessel region from the image obtained in the pre-scan;
setting a velocity encoding value for a main scan based on the maximum blood velocity;
executing the main scan of the subject by applying the velocity encoding having the set value; and
outputting an image showing the blood velocity in the target blood vessel region obtained in the main scan.

13. A non-transitory, computer-readable tangible recording medium which records thereon, a program for causing, when read by a computer, the computer to execute the method for operating a magnetic resonance imaging apparatus according to claim 12.
